(19) United States
(12) Patent Application Publication
(10) Pub. No.: US 2010/0248298 A1
(43) Pub. Date: Sep. 30, 2010

(54) MASS SPECTROMETRIC IDENTIFICATION OF MICROORGANISMS IN COMPLEX SAMPLES

(75) Inventors: Markus Kostrzewa, Taucha (DE); Thomas Maier, Leipzig (DE); Stefan Klepel, Taucha (DE)

Correspondence Address: LAW OFFICES OF PAUL E. KUDIRKA, 40 BROAD STREET, SUITE 300, BOSTON, MA, 02109, US

(73) Assignee: BRUKER DALTONIK GMBH, Bremen (DE)

(21) Appl. No.: 12/695,235

(22) Filed: Jan. 28, 2010

(30) Foreign Application Priority Data
Feb. 3, 2009 (DE) 10 2009 007 266.7

(51) Int. Cl. C12Q 1/04

(52) U.S. Cl. 435/34

(57) ABSTRACT

Microorganisms are identified as present in a complex sample or mixed culture by acquiring a mass spectrum of the sample and comparing it to combination spectra, each of which is formed by combining at least two reference mass spectra of known microorganisms. Microorganisms corresponding to the reference spectra used to form the combination spectrum are identified as present in the sample if that combination spectrum exhibits a better match with the sample mass spectrum than any one of reference mass spectra used to form that combination spectrum. It is also possible to identify microorganisms by forming a difference spectrum by subtracting a reference mass spectrum from the sample mass spectrum and comparing the difference spectrum to the reference mass spectra.

### BACKGROUND

This invention relates to the mass spectrometric identification of microorganisms in complex samples with mixed cultures. The rapid, error-free identification of microorganisms plays a prominent role in food analysis, monitoring and control of biotechnological processes, detection of biological warfare, and particularly in clinical microbiology. Microorganisms, which are also called germs or microbes, are usually microscopically small organisms which include bacteria, fungi (e.g. yeasts), microscopic algae, and protozoa (e.g. plasmodia, as malaria pathogens). Just for the purpose of identification, viruses may also be included here, although these, due to the lack of any metabolism, do not count as living beings.

The identification of a microorganism involves classifying it into the following taxonomic hierarchical levels: domain, kingdom, phylum, class, order, family, genus, species, subspecies. The terms “strain” and “isolate” used below describe a population of microbes which is multiplied from a single organism or which derives from a pure culture (isolate). The individual organisms of a strain or isolate are genetically identical. The identification of a microorganism always relates to the identification using a strain or an isolate. For a person specialized in the field, the term “microorganism” is often synonymous with the term “strain” or “isolate”.

The traditional identification of microorganisms in a sample under investigation requires the culturing of colonies of the microorganisms. The Analytical Profile Index (API) used in laboratory practice comprise different culture media, which can be used to detect specific metabolic characteristics of the microorganisms, thus allowing a first rough taxonomic classification. Moreover, the microscopic morphology of individual organisms of a colony and the morphology of the colony itself are investigated. On the other hand, new identification methods based, for example, on the replication of specific genetic sequences by polymerase chain reaction (PCR) or on a mass spectrometric detection of specific molecular cell components of microorganisms, have been known for some years. These new methods are superior to conventional methods in terms of specificity, error rates and analysis time.

The identification of bacteria by mass spectrometric detection methods has been described in detail in a review by van Baar (FEMS Microbiology Reviews, 24, 2000, 193-219: “Characterization of bacteria by matrix-assisted laser desorption/ionization and electrospray mass spectrometry”). The identification is achieved by means of a similarity analysis between a mass spectrum of the sample under investigation and reference spectra of known bacteria. The similarity analysis assigns a similarity indicator to each reference spectrum. This similarity indicator is a measure of how closely each reference spectrum matches the mass spectrum of the sample. A bacterium, or more generally a microorganism, may be regarded as identified in a sample if the similarity indicator of the most similar reference spectrum is greater than a specified limit value, for example.

In practice, a simple and low-cost method for the mass spectrometric identification of microorganisms based on MALDI (matrix-assisted laser desorption/ionization) time-of-flight mass spectra has gained wide-spread acceptance by microbiologists. The microorganisms of a sample under investigation are usually cultivated on an agar plate with a culture medium for several hours to obtain pure cultures for each microorganism frequently contained in the sample. A few hundred microbes from such a colony are transferred as intact cells onto a sample support, where they are sprinkled with a conventional matrix solution for ionization by matrix-assisted laser desorption (MALDI). The organic solvent in which the matrix substance is dissolved, and the added acid usually destroy the walls of the cells on the sample support, releasing molecular cell components from the interior of the cell, in particular soluble proteins. The organic solvent subsequently evaporates and the matrix substance crystallizes; the released molecular cell components are incorporated into the crystallized matrix layer as analyte molecules. The MALDI sample thus prepared is introduced into a time-of-flight mass spectrometer and bombarded with laser pulses, causing the analyte molecules together with the matrix substance to be desorbed and partially ionized. The ions of the analyte molecules (analyte ions) are accelerated in an electric field and impinge on a detector after mass-dependent flight times. The flight times measured with the detector are converted into masses (more accurately: into mass-to-charge ratios) using known calibration functions.

The mass spectra of microorganisms are very specific to the respective microorganism because even minor genetic differences between different species, sub-species and even strains are reflected in the masses of the expressed proteins and thus also in the signals of the mass spectrum. In order to identify microorganisms, a mass spectrum of the sample is acquired and compared with mass spectra of known microorganisms, which are available as reference spectra in a library. The similarity analysis can be made visually by a person, but is preferably done with the aid of a computer. If the library contains neither a reference spectrum of the microorganism itself nor one of a microorganism of the same species (which may occur due to the enormous number of species and the limited size of current libraries), a computer-aided similarity analysis can nevertheless enable assignment to higher taxonomic classification levels (e.g. at genus or family level) because there is a high probability for at least some proteins to be identical for related microorganisms. If the library does not contain a reference spectrum of the strain itself, but reference spectra of other strains of the same species, a correct identification of the species is highly probable.

In addition to the specificity of the mass spectra, the reproducibility of the mass spectra under changed culturing and sample preparation conditions, and the associated error rate, are of crucial importance in actual practice. It has become apparent that although the intensity values of the signals in the mass spectra of microorganisms may strongly fluctuate, the mass values of the signals in the mass spectra have a very high reproducibility, in fact irrespective of the particular culture medium, the conditions for cultivation of the colonies and the age (degree of maturity) of the colonies. The error rate of mass spectrometric identification has proven to be much lower than that of conventional identification methods. It was demonstrated that, by using libraries with many hundreds of species and several thousand strains, by far over 99% of the species are correctly identified (true positive identification: >99%, true negative identification: 100%). It was also found that the actual error rate is sometimes difficult to determine because in quite a few cases the reference spectra in the libraries were wrongly designated, due to a false identification of the strains delivered to measure the reference spectra. Ultimately, in doubtful cases, only genetic sequencing helps to decide on an unequivocal correct identification.

In the publication by Jarman et al. (Analytical Chemistry, 72(6), 2002, 1217-1223: “An Algorithm for Automated Bacterial Identification Using Matrix-Assisted Laser Desorption/Ionization Mass Spectrometry”) computer-assisted methods for automated generation of reference spectra and also for similarity analysis between a mass spectrum of a sample under investigation and the automatically generated reference spectra have been reported.

In Jarman et al. the reference spectra are no longer measured mass spectra, but spectral fingerprints, which are derived from several measured mass spectra (repeat spectra) by means of a statistical algorithm. A repeat spectrum can be based on a technical or a biological repetition: in a technical repetition, the microorganisms in the sample itself are unchanged. A technical repeat spectrum in a MALDI time-of-flight mass spectrometer usually arises simply because several laser pulses are required for a measured mass spectrum. Further technical repeat parameters are, for example, the position of the laser focus on the sample surface, the laser energy, the sample quantity, the matrix used and/or the type of sample preparation (dried droplet, thin layer and multiple preparation). Biological repeat refers to the culturing conditions (culture medium, temperature) and the age of the colony, for example. A spectral fingerprint according to Jarman et al. contains the following values of the signals averaged over the measured repeat spectra: the averaged mass, the variance of the mass, the average intensity, the variance of the intensity and a percentage value indicating how frequently a signal occurs in the repeat spectra. As a rule, only those signals which exhibit a predetermined minimum number of occurrences in the repeat spectra are taken into account for a spectral fingerprint (reference spectrum). In this way all noise signals, which interfere with identification, are removed without reducing the specificity of the spectral fingerprint.

The similarity analysis between the mass spectrum of a sample under investigation and one of the spectral fingerprints in the library is done by determining for every signal of the mass spectrum a probability that it corresponds to a reference signal of the spectral fingerprint, the distance from the corresponding reference signals and also their variance being taken into consideration. The individual probabilities of the signals of the mass spectrum thus obtained are then used to derive an similarity indicator for the agreement with the spectral fingerprint (reference spectrum). The reference spectra are usually ordered according to their agreement with the mass spectrum, and the designations of the microorganisms which are assigned to the ordered reference spectra are shown with the corresponding similarity indicators in a list of results.

In the publication of Jarman et al., complex samples are also produced in a blind trial by mixing 2 or 3 known microorganisms from pure cultures, Jarman et al. show that, with the method described, the microorganisms present in the complex sample can be identified if the identification criterion is taken to be that the similarity indicators of the reference spectra are greater than a certain limit value. However, the library used there consisted only of the reference spectra of 5 different non-related microorganisms, so the findings can only be applied to current library sizes to a limited extent. Moreover, there are indications that the error rates (false-positive, and also false-negative) increase greatly if complex samples contain more than three different microorganisms.

Thus, in laboratory practice with large libraries, there is still a great need to identify complex samples with certainty in order to avoid a time-consuming second cultivation when a mixed culture is suspected; this is of crucial importance particularly in the clinical diagnosis of pathogens. Furthermore, from the patent application DE 10 2007 058 516.2-54, a method has been reported where the pathogens are identified directly from bodily fluids using MALDI-TOF mass spectra, which allows the analysis time to be shortened considerably. Samples which are obtained directly from bodily fluids are, however, sometimes complex samples, i.e. mixtures with more than one type of microorganism, which complicates their identification.

### SUMMARY

The identification method according to the invention comprises combining at least two reference spectra to form a combination spectrum and extending a similarity analysis to the combination spectrum. The microorganisms belonging to the combination spectrum are regarded to be identified in the sample, for example, if the combination spectrum exhibits a better match with the mass spectrum of the sample than any one of the reference spectra and if the similarity indicator is greater than a predefined minimum value. The expression “microorganisms of a combination spectrum” encompasses those microorganisms whose assigned reference spectra are combined to form the combination spectrum. It is within the scope of the invention that more than three reference spectra are combined to form different combination spectra and that only the microorganisms of those combination spectra are regarded as identified in the sample which have a better match with the mass spectrum of the sample than any one of the reference spectra or any one of their respective reference spectra.

In combination spectra, the microorganisms should differ in respect of their species, genus and/or a higher taxonomic hierarchy level.

The method according to the invention can be used to identify microorganisms in complex samples, which significantly reduces the analysis time for a sample containing a mixture of microorganisms because a special isolating cultivation is made superfluous. Samples obtained directly from bodily fluids can be reliably identified, for example, even when the presence of a mixture of two or three microbes cannot be excluded.

The reference spectra used in the method according to the invention are mass spectra of known microorganisms usually stored in a computer-based database. The term “mass spectrum” encompasses both measured as well as processed mass spectra, where the processing can involve the summation of repeat spectra, correction of the background, calibration of the mass axis or noise suppression, for example. A mass spectrum as defined in this invention may have the form of a so-called peak list, which contains only the masses and intensities of the signals present in a mass spectrum, or of a spectral fingerprint known from the prior art, which is determined from repeat spectra and which may have a reduced number of signals specific to the respective microorganism. A “known microorganism” is classified in the taxonomic hierarchy levels; usually not only the name of the species or subspecies, but also an unambiguous strain designation is given. A reference spectrum is usually highly specific for the assigned microorganism.

Reference spectra can be combined to form a combination spectrum by simply forming an unweighted or weighted average of the reference spectra, for example. The weighting of the reference spectra can be iteratively optimized so that a maximum agreement with a highest similarity indicator is achieved between the combination spectrum and the mass spectrum of the sample. If the reference spectra are available as lists (e.g. as peak lists), the lists can be combined and sorted according to masses. If the masses of some signals in the lists are the same, or the masses agree within a predefined tolerance range, these “identical” signals can be combined in the combination spectrum in each case to form one signal. If the reference spectra are the spectral fingerprints known from the prior art, a combination spectrum can be generated so that the statistical algorithm with which a spectral fingerprint is calculated is applied to all repeat spectra of the combined reference spectra, or so that the corresponding averaged values of the combined reference spectra themselves are averaged. The number of signals in a combination spectrum is greater than the number of signals in each of the individual reference spectra that are combined to form the combination spectrum.

The method according to the invention is preferably integrated into a hierarchical procedure where, in a first step, a usual similarity analysis is performed and the results are used to perform the method according to the invention. At first, all the reference spectra of the known microorganisms are compared (by calculation of the similarity indicators) with a mass spectrum of the sample under investigation, and a ranked list of the reference spectra according to their agreement with the mass spectrum of the sample is presented. A subset of the “best” reference spectra (best set) is derived from the ranking.

The best set can be selected by the user from the ranking of the reference spectra after the similarity analysis of the first step. The best set can also be determined automatically, for example, in such a way that it consists of a specified number of the best reference spectra of the ranking. The best set can consist of the best 3 to 20 reference spectra of the ranking or of the best 0.01% to 0.1% of the ranking, for example. A further automatic selection includes in the best set all reference spectra whose similarity indicator is larger than a specified minimum value.

In a second step, combination spectra of all taxonomically different microorganisms from the best set are formed. The combinations may contain two reference spectra each, or even three, if that many taxonomically different microorganisms are present in the best set. If the microorganisms of the best set do not differ taxonomically, the procedure is halted because a mixed culture cannot be assumed.

In a third step, the similarity analysis is extended to the combination spectra, and the indicators for the similarities of the combination spectra with the sample spectrum are calculated. The microorganisms of the best combination spectrum are regarded to be identified in the sample if the best combination spectrum has a better match with the mass spectrum of the sample than any one of the reference spectra of the best set and supersedes a given minimum value for the similarity indicator.

It is within the scope of the invention that, in the hierarchical method, more than two or three reference spectra of the best set are combined to form different combination spectra, at least two microorganisms of each combination spectrum being taxonomically different. The microorganisms of those combination spectra which have a better match with the mass spectrum of the sample than any of their respective reference spectra are regarded as identified in the sample. On the other hand, only the microorganisms of those combination spectra which have a better match with the mass spectrum of the sample than any one of the reference spectra of the best set, or whose similarity indicator is greater than a specified minimum value, can be regarded as identified in the sample. The reference spectra of the best set are preferably combined in pairs to form the different combination spectra. The taxonomic diversity of the microorganisms whose reference spectra are combined to form a combination spectrum refer at least to their species and/or their genus.

If certain microorganisms often occur together in samples, the combination spectra of the corresponding microorganisms can also be readily stored as additional reference spectra in the database. In this case, the similarity indicators of the stored combination spectra, which result from a similarity analysis with a mass spectrum of a sample under investigation, can be used as the criterion for identification of the microorganisms or as the criterion for the presence of a mixed culture.

There is also a further method according to the invention to identify microorganisms in complex samples. It involves subtracting at least one reference spectrum from the mass spectrum of a sample under investigation, i.e. a difference spectrum is generated from the sample spectrum instead of a combination of reference spectra. The resulting difference spectrum is again compared with the reference spectra by a similarity analysis. A microorganism is only regarded as identified if its reference spectrum sufficiently matches the difference spectrum, e.g. if the similarity indicator of the difference spectrum is greater than a specified minimum value. In order to identify more than one microorganism in a sample, different difference spectra are generated and those microorganisms whose reference spectra sufficiently match one of the difference spectra are regarded as identified in the sample. The reference spectra used to generate the difference spectra can be selected from a best set, analogously to the hierarchical procedure described above.

As already explained in the Prior Art, MALDI time-of-flight mass spectra are well-established in the practice of mass spectrometric detection. However, in addition to ionization by matrix-assisted laser desorption, other types of ionization are also suitable in principle for the method according to the invention, e.g. electrospray ionization (ESI) or desorption methods with subsequent chemical ionization (CI). Moreover, different types of mass analyzer can be used, e.g. time-of-flight mass spectrometers with axial or orthogonal ion injection, ion trap mass spectrometers or quadrupole filters.

### BRIEF DESCRIPTION OF THE DRAWINGS

FIG. 1 is a schematic representation of a first method according to the invention for identifying bacteria in complex samples with the steps S0, S11 and S12.

FIG. 2 is a schematic representation of a second method according to the invention with a hierarchical method sequence and the steps S0, S21, S22, S23 and S24.

FIG. 3 shows a normalized, measured MALDI time-of-flight mass spectrum (10) of a sample under investigation and a reconstructed mass spectrum (20) derived from a peak list.

FIGS. 4A and 4B show two reference spectra (30) and (40) of a best set (REF_{B}), which both sufficiently match the mass spectrum (20) of the sample, which is also shown, in order for the corresponding microorganisms (FIG. 3A: Pseudomonas aeruginosa; FIG. 3B: Proteus mirabilis) to be regarded as identified in the sample. The microorganisms differ from each other as from their taxonomic order.

FIG. 5 shows the mass spectrum of sample (20) and a combination spectrum (50) of two reference spectra of the best set (REF_{B}), where one of the reference spectra originates from a microorganism of the species “Pseudomonas aeruginosa” and the other reference spectrum from a microorganism of the species “Proteus mirabilis”.

FIG. 6 is a schematic representation of a third method according to the invention consisting of the steps S0, S31 and S32 and using difference spectra rather than combination spectra to identify microorganisms in complex samples.

### DETAILED DESCRIPTION

While the invention has been shown and described with reference to a number of embodiments thereof, it will be recognized by those skilled in the art that various changes in form and detail may be made herein without departing from the spirit and scope of the invention as defined by the appended claims.

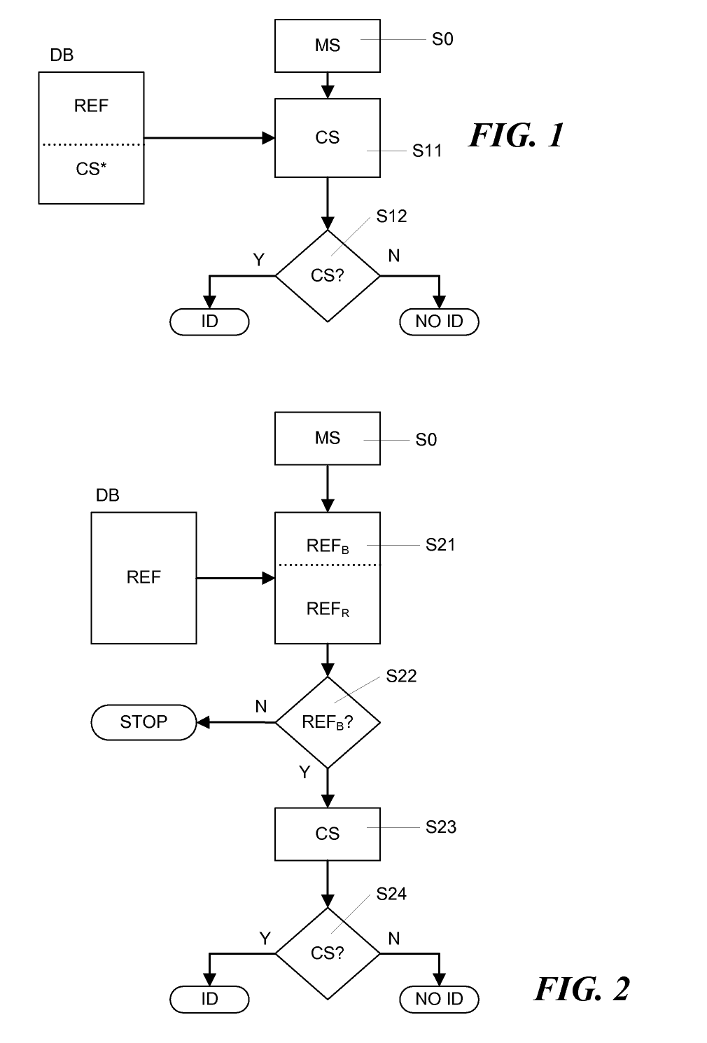

A method according to the invention for identifying bacteria in complex samples is schematically presented in FIG. 1. In step S0, a sample under investigation is cultured, and then a MALDI time-of-flight mass spectrum of the sample (MS) is acquired. In step S11, at least two reference spectra (REF) of a database (DB) are combined to form a combination spectrum, or a combination spectrum (CS*) previously stored in the database (DB) is selected. In step S12, the microorganisms of the generated or selected combination spectrum (CS) are only regarded as identified in the sample (ID) if the combination spectrum (CS) fulfils one of the above-stated criteria, e.g. a better match with the mass spectrum of the sample (MS) than any of the reference spectra from which the combination spectrum (CS) was generated. Otherwise, the corresponding microorganisms of the combination spectrum (CS) are regarded as not identified (NO ID).

A particularly preferred hierarchical method for identifying bacteria in complex samples is schematically presented in FIG. 2, showing the steps S0 and S21 to S24 described below.

In step S0, a sample under investigation is transferred onto a mass spectrometric sample support. The sample is assumed to be, with some probability, a mixture of two or more microbes, either because the colonies on an agar plate were not clearly separated, or the microbes came directly from a blood culture or a culture of other body fluids. The mass spectrometric sample support usually has a large number of spatially separated sample points, onto each of which microbe samples can be loaded. The microbe sample on the sample support is sprinkled with a solution of a conventional matrix substance for ionization by matrix-assisted laser desorption (MALDI). The organic solvent usually penetrates into the transferred cells and destroys them. The solvent subsequently evaporates and the dissolved matrix substance crystallizes, while some of the molecular cell components, particularly soluble proteins, released by the destruction of the cells are incorporated as analyte molecules into the matrix crystals.

The matrix crystals and the analyte molecules incorporated therein are bombarded with laser light pulses in the ion source of a time-of-flight mass spectrometer, causing analyte molecules to be desorbed and ionized together with the matrix substance. The analyte ions thus produced are temporally separated in the time-of-flight mass spectrometer due to their mass-dependent time of flight, and are detected in a detector. The measured flight times of the ions are then converted into masses. The overwhelming majority of ions are protein ions which, after the ionization by the MALDI process, are present as singly charged ions (charge number z=1), which is why we can simply refer here to the mass m of the analyte ion, instead of using the correct term “charge-related mass” m/z.

The time-of-flight mass spectrometers used for the identification of microorganisms are operated without a reflector because the detection sensitivity then is much higher (“linear operating mode”), although the mass resolution and the mass accuracy are significantly better when using a reflector. But in the reflector mode, only around a twentieth of the ion signals appear, and the detection sensitivity is one to two orders of magnitude worse. The high sensitivity is due to the fact that, in linear mode, not only the stable analyte ions, but also fragment ions and even the neutral particles from metastable decays of the analyte ions are detected. The secondary electron multipliers (SEM) used as the detector detect not only the analyte and fragment ions but even the neutral particles which are created from ion disintegrations during the time of flight, because these neutral particles also generate secondary electrons when they strike the SEM. When a singly charged molecular ion decays into five particles, then necessarily four of them are neutrals. All the fragment ions and neutral particles of one species of molecular ion possess the speed of the molecular ion from which they originated, and therefore reach the detector simultaneously with their molecular ions, creating an increased ion signal. The increased detection sensitivity is often so crucial for the identification of microorganisms that the disadvantages of the linear mode of operation must be tolerated. For this application, one even increases the energy of the desorbing and ionizing laser pulses, which leads to an increased yield of analyte ions, but also strongly increases the number of fragment particles per molecular ion, which is not a problem here for the reasons stated.

The acquisition of a mass spectrum with a time-of-flight mass spectrometer usually requires the acquisition of many individual spectra, which are each generated by a single laser pulse and usually added together to form a sum spectrum by adding measurement points with the same flight time. In general, a sum spectrum consists of several hundred individual spectra, for which modern time-of-flight mass spectrometers need only a few seconds. Such a sum spectrum is usually processed further: for example, the time of flight is converted into a mass by a function calibrated before, the background is corrected and the noise in the mass spectrum is filtered out. A peak list is usually generated from the processed sum spectrum.

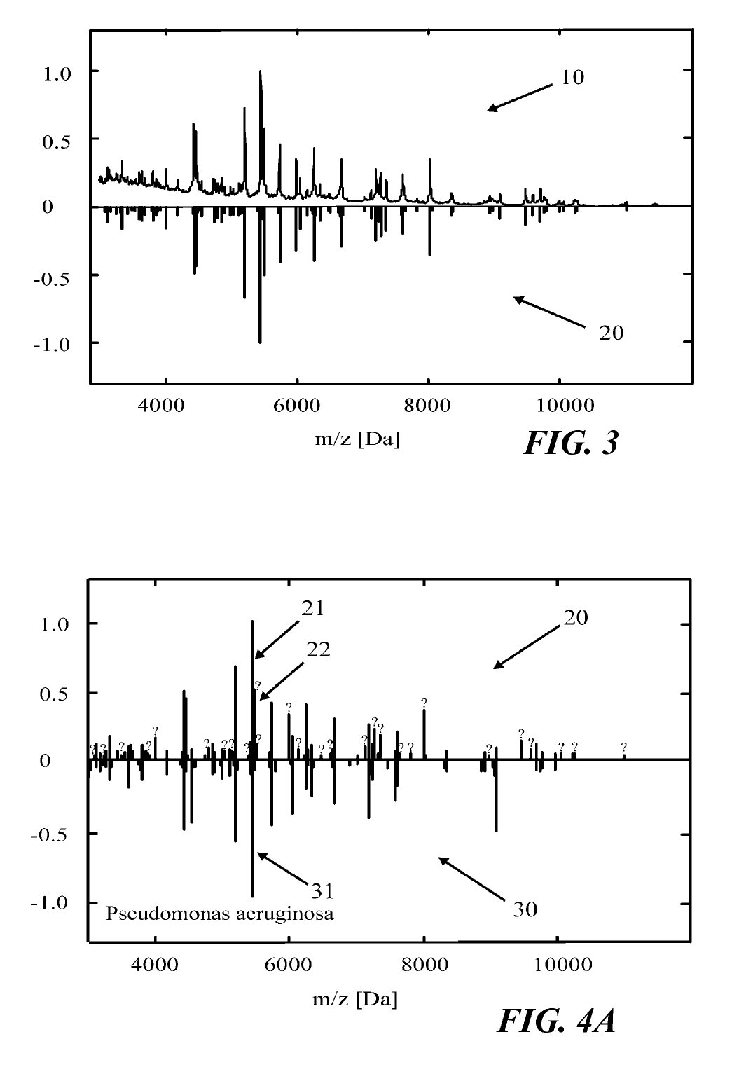

The upper half of FIG. 3 shows a measured MALDI time-of-flight mass spectrum (10) of a sample under investigation, which has been normalized to the value 1. The lower half of FIG. 3 shows a spectrum reconstructed from the peak list (20) of the sum spectrum (10), which contains only the significant signals of the sum spectrum (10) and thus requires much less storage space than the sum spectrum (10). The mass axis in FIG. 3 ranges from 3,000 to 12,000 daltons. At present, the time-of-flight mass spectra used for the identification of microorganisms are usually acquired in a mass range between around 2,000 daltons and 20,000 daltons. The signals in the lower mass range up to about 2,500 daltons are not very usable. For ionization by the MALDI process, the signals in the lower mass range are predominantly attributable to ions of the matrix substance and their clusters, but also to those molecular cell components that vary depending on the culturing and preparation conditions, and are therefore not suitable for a reliable identification. The best identification results are obtained if only the signals in the mass range between 3,000 and 15,000 daltons are evaluated.

In step S21, the measured mass spectrum (MS), here the peak list, on which the spectrum (20) is based, is compared with all the reference spectra (REF) which are stored as peak lists in the database (DB). Each comparison is done by a calculation of the similarity indicators. The reference spectra (REF) were obtained previously from pure cultures (isolates) of correctly identified microorganisms. Nowadays, validated commercial databases for the identification of microorganisms contains reference spectra of several thousand different microorganisms.

Table 1 shows the genus, species and strain designations together with the similarity indicators of the twenty microorganisms whose reference spectra best match the mass spectrum of the sample (MS), i.e. of the peak list for spectrum (20).

TABLE 1

| | | |
|---|---|---|
| 1 | Pseudomonas aeruginosa 8147_2_CHB | 2.373 |
| 2 | Pseudomonas aeruginosa DSM 50071T HAM | 2.325 |
| 3 | Proteus mirabilis DSM 50903_DSM | 2.257 |
| 4 | Pseudomonas aeruginosa ATCC 27853_CHB | 2.208 |
| 5 | Pseudomonas aeruginosa 19955_1 CHB | 2.189 |
| 6 | Pseudomonas aeruginosa ATCC 27853 THL | 2.187 |
| 7 | Proteus mirabilis 13210 1_CHB | 2.185 |
| 8 | Proteus mirabilis (PX) 22086112_MLD | 2.090 |
| 9 | Proteus mirabilis 9482_2 CHB | 2.085 |
| 10 | Proteus mirabilis DSM 18254_DSM | 2.084 |
| 11 | Proteus mirabilis 22086103_MLD | 2.071 |
| 12 | Proteus mirabilis DSM 30115_DSM | 2.064 |
| 13 | Proteus mirabilis DSM 46227_DSM | 2.059 |
| 14 | Proteus mirabilis DSM 788_DSM | 2.028 |
| 15 | Pseudomonas jinjuensis LMG 21316 HAM | 1.671 |
| 16 | Proteus penneri DSM 4544_DSM | 1.556 |
| 17 | Pseudomonas resinovorans LMG 2274 HAM | 1.426 |
| 18 | Proteus vulgaris DSM 13387 HAM | 1.422 |
| 19 | Serratia rubidaea DSM 46275 DSM | 1.299 |
| 20 | Proteus vulgaris (PX) 22086129_MLD | 1.283 |
| . . . | | |

The similarity indicator in the right-hand column of Table 1 is a logarithmic parameter normalized to a maximum value of 3.00 for a complete agreement with the peak list (20). An similarity indicator between 2.3 and 3 is a criterion for a very safe identification of the species. With an similarity indicator of between 2.0 and 2.3, a reliable identification of the genus and a probable identification of the species is assumed. The range of values between 1.7 and 2.0 still allows a probable identification of the genus, while below a value of 1.7 no reliable identification has been obtained.

In this example embodiment, the first ten microorganisms of Table 1 are arbitrarily selected as best set of reference spectra (REF_{B}), which are used in the steps S22 and S23 below. The remaining reference spectra (REF_{R}) of the database are not considered further in the following steps.

In step S22, the investigation focuses on whether the selected microorganisms of the best set differ in respect of their species, genus and/or a higher taxonomic hierarchy level. If all the microorganisms selected are only different strains of the same species, the procedure is halted. If not, the procedure is continued with steps S23 and S24.

The best set of reference spectra contains exactly two species of microorganisms: Pseudomonas aeruginosa and Proteus mirabilis. Both microorganisms belong to the taxonomic class “Gammaproteobacteria”, but already differ by their taxonomic orders “Pseudomonadales” and “Enterobacteriales”. The best set contains five reference spectra of microorganisms of the species “Pseudomonas aeruginosa” and five reference spectra of microorganisms of species “Proteus mirabilis”; even the “best” microorganism from the best set (Pseudomonas aeruginosa 8147_{—}2_CHB) has an similarity indicator which is only slightly greater than 2.3. Pseudomonas aeruginosa is a gram-negative oxidase-positive bacterium of the genus Pseudomonas (family: Pseudomonadaceae, order: Pseudomonadales). This widespread bacterium is found in humid environments and, as a pathogen, it plays a significant role in the increasing and often life-threatening hospital infections because it has multiple resistances to antibiotics due to its metabolism and the structure of its cell membrane. Proteus mirabilis is a gram-negative rod-shaped bacterium of the genus Proteus (family: Enterobacteriaceae, order: Enterobacteriales). This bacterium is a facultative pathogen which often occurs in the large intestine even of healthy people, but does not necessarily cause diseases. It can, however, cause additional clinical syndromes in immunodeficient persons, but treatment with antibiotics is usually successful.

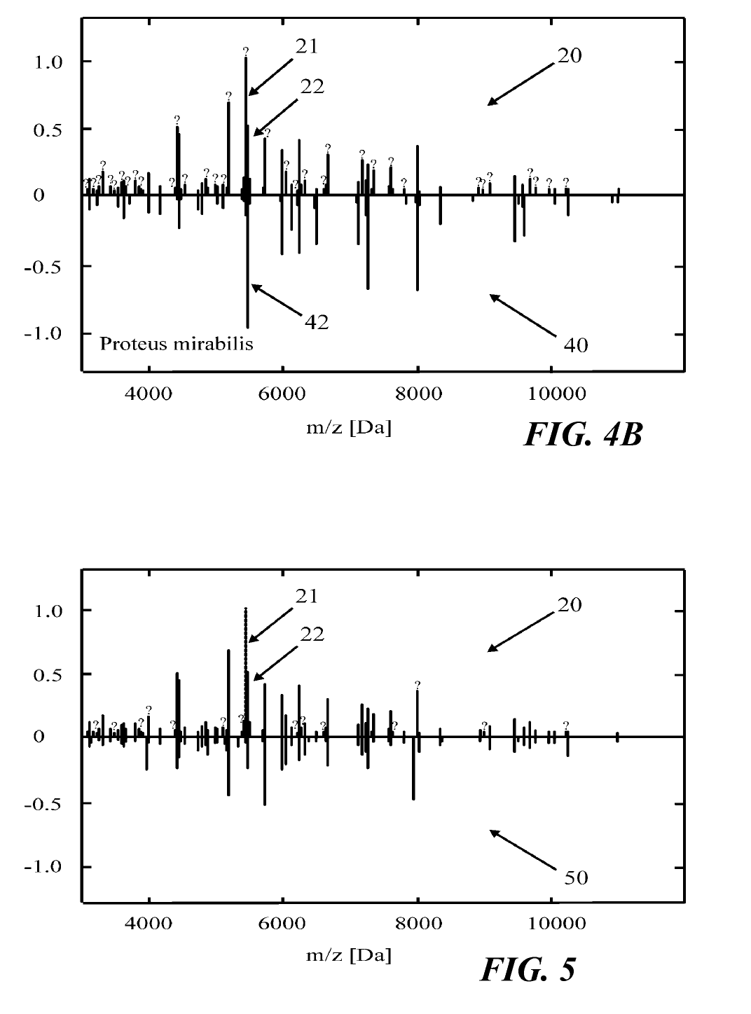

FIGS. 4A and 4B show the reconstructed reference spectra of “Pseudomonas aeruginosa 8147_{—}2_CHB” (30) and “Proteus mirabilis DSM 50903_DSM” (40), each in comparison with the spectrum of peak list (20) of the sample under investigation. The two reference spectra (30) and (40) exhibit the highest similarity indicators of their relevant species (see rows 1 and 3 in Table 1). In FIGS. 4A and 4B those signals of the peak list (20) which do not occur in the reference spectrum (30) or in the reference spectrum (40) are annotated with a question mark. While the signal (21) of peak list (20) appears in the reference spectrum (30) as signal (31), for example, there is nothing corresponding to signal (22) in the reference spectrum (30). The reference spectrum (40) has, in contrast, a signal (42) which corresponds to signal (22). The two reference signals (30) and (40) appear to complement each other, at least with reference to the signals (21) and (22), which points to a mixed culture and could explain the large number of annotated signals in FIGS. 4A and 4B.

For such a sample, where a widespread pathogen of hospital infections is contained in the results list alongside a merely facultative pathogen, rapid and certain identification is particularly important to ensure successful treatment.

In step S23, the reference spectra of the different species of the best set (REF_{B}) are combined in all possible pairs to form different combination spectra (CS). From the 10 reference spectra (5× “Pseudomonas aeruginosa”, 5× “Proteus mirabilis”) a total of 25 (5×5) combination spectra (CS) are formed, which are again compared with the peak list (20). For reasons of clarity, Table 2 shows only the best 10 of the 25 pairs of microorganisms with the similarity indicators of the corresponding combination spectrum.

TABLE 2

| | | |
|---|---|---|
| 1 | Pseudomonas aeruginosa DSM 50071T HAM + | 2.640 |
| | Proteus mirabilis 13210 1_CHB | |
| 2 | Pseudomonas aeruginosa DSM 50071T HAM + | 2.575 |
| | Proteus mirabilis 9482_2 CHB | |
| 3 | Pseudomonas aeruginosa DSM 50071T HAM + | 2.565 |
| | Proteus mirabilis DSM 50903_DSM | |
| 4 | Pseudomonas aeruginosa ATCC 27853_CHB + | 2.565 |
| | Proteus mirabilis 13210 1_CHB | |
| 5 | Pseudomonas aeruginosa DSM 50071T HAM + | 2.559 |
| | Proteus mirabilis DSM 18254_DSM | |
| 6 | Pseudomonas aeruginosa 8147_2_CHB + | 2.549 |
| | Proteus mirabilis 13210 1_CHB | |
| 7 | Pseudomonas aeruginosa 8147_2_CHB + | 2.547 |
| | Proteus mirabilis 9482_2 CHB | |
| 8 | Pseudomonas aeruginosa ATCC 27853_CHB + | 2.545 |
| | Proteus mirabilis 9482_2 CHB | |
| 9 | Pseudomonas aeruginosa ATCC 27853_CHB + | 2.533 |
| | Proteus mirabilis DSM 50903_DSM | |
| 10 | Pseudomonas aeruginosa 8147_2_CHB + | 2.513 |
| | Proteus mirabilis DSM 50903_DSM | |
| . . . | | |

FIG. 5 shows the spectrum of the peak list (20) and the combination spectrum (50), which was combined from the reference spectra of “Pseudomonas aeruginosa DSM 50071T HAM” and “Proteus mirabilis 13210 1_CHB” (No. 1 in Table 2). The combination spectrum (50) shows the best match of all 25 combination spectra (CS) and has an similarity indicator which is significantly greater than 2.3. Correspondingly, the number of non-matching signals, which are annotated in the peak list (20) with a question mark, is significantly smaller than for the best reference spectra (30) and (40).

In the concluding step S24 of FIG. 2, each of the 25 combination spectra (CS) is investigated to see whether it shows a better match with the peak list (20) than the two reference spectra (REF_{B}) from which it was combined. If this is the case, the corresponding microorganisms of the combination spectrum (CS) are regarded as identified (ID) in the sample. Otherwise, the sample is labeled not identifiable (NO ID). It can be seen that here, the above-mentioned criterion for an identification is fulfilled, so both species “Pseudomonas aeruginosa” and “Proteus mirabilis” are regarded as identified. It should be noted that the similarity indicators of all the combination spectra (CS) are greater than 2.3, which additionally speaks for a very probable identification of both species. A comparison of Tables 1 and 2 makes clear that the combination spectrum from the two best reference spectra of both species (entries 1 and 3 in Table 1) do not show the largest similarity indicator, but are only in tenth place in the ranking.

It is within the scope of the present invention that the reference spectra of the best set can be specified in other ways and combined in different ways to form a combination spectrum or several combination spectra. The criterion for the identification of the microorganisms used in this example embodiment is also only one preferred possibility.

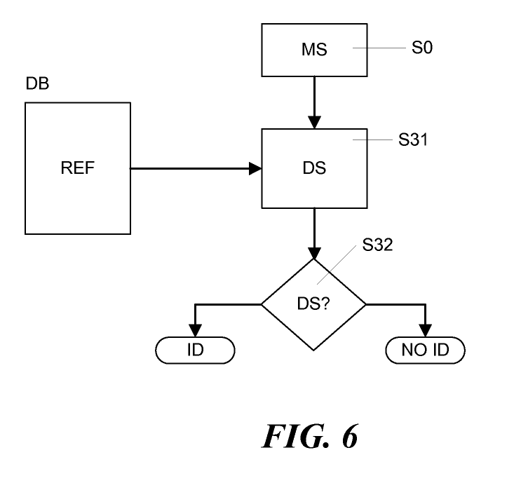

FIG. 6 is a schematic representation of a further preferred method as defined in the present invention. Step S0 corresponds to the one in FIGS. 1 and 2. In step S31, a difference spectrum (DS) is generated by subtracting at least one reference spectrum (REF) of a database (DB) from the mass spectrum (MS). In step S32, the difference spectrum is compared with the reference spectra (REF) and analyzed to see whether it matches one of the reference spectra (REF) sufficiently well for the corresponding microorganism to be identified with certainty.

The features according to the invention detailed in the description of the invention, in the example embodiments and in the figures can each be applied individually or as a combination of several features in order to achieve the objective.

What is claimed is:

1. A method for identifying microorganisms present in a sample, comprising:
(a) acquiring a mass spectrum of the sample;
(b) comparing the sample mass spectrum to each of a plurality of reference mass spectra, wherein each reference mass spectrum is a mass spectrum of a known microorganism;
(c) selecting as a best set of reference mass spectra, those reference mass spectra that are found to most closely match the sample spectrum in the comparisons in step (b);
(d) combining reference mass spectra of microorganisms of different species in the best set of reference mass spectra to form combination spectra; and
(e) comparing the sample mass spectrum to the combination spectra.

2. The method of claim 1, wherein selected microorganisms are identified as present in the sample when a match between the sample mass spectrum and a combination spectrum formed from reference mass spectra of the selected microorganisms is better than any match between the sample spectrum and any single reference mass spectrum of the reference mass spectra combined to form the combination spectrum.

3. The method of claim 1, wherein step (d) comprises:
(d1) combining reference mass spectra with a weighting factor for each reference mass spectrum to form a combination spectrum;
(d2) determining a similarity indicator between the combination spectrum formed in step (d1) and the sample mass spectrum; and
(d3) repeating steps (d1) and (d2) while modifying the weighting factors until the similarity indicator is maximized.

4. The method of claim 1, wherein selected microorganisms are identified as present in the sample when a match between the sample mass spectrum and a combination spectrum formed from reference mass spectra of the selected microorganisms is better than any match between the sample spectrum and any single reference mass spectrum in the best set of reference spectra.

5. The method of claim 1, wherein selected microorganisms are identified as present in the sample when a similarity indicator for a match between the sample mass spectrum and a combination spectrum formed from reference mass spectra of the selected microorganisms is greater than a predetermined minimum value.

6. The method of claim 1, wherein in step (c), reference mass spectra are selected manually to form the best set of reference mass spectra.

7. The method of claim 1, wherein step (c) comprises ranking each reference mass spectra by closeness of a match between that reference mass spectra and the sample mass spectra and selecting a predefined number of highest ranking reference mass spectra to form the best set of reference mass spectra.

8. The method of claim 7, wherein the predefined number is between 3 and 20.

9. The method of claim 1, wherein step (c) comprises ranking each reference mass spectra by closeness of a match between that reference mass spectra and the sample mass spectra and selecting 0.01 to 0.1 percent of highest ranking reference mass spectra to form the best set of reference mass spectra.

10. The method of claim 1, wherein step (c) comprises computing for each reference mass spectra a similarity factor that indicates the closeness of a match between that reference mass spectra and the sample mass spectra and selecting reference mass spectra with a similarity indicator greater than a predetermined minimum value as the best set of reference mass spectra.

11. A method for identifying microorganisms present in a sample, comprising:
(a) acquiring a mass spectrum of the sample;
(b) obtaining a plurality of reference mass spectra, wherein each reference mass spectrum is a mass spectrum of a known microorganism;
(c) combining reference mass spectra of microorganisms of different species to form combination spectra; and
(d) comparing the sample mass spectrum to the combination spectra.

12. The method of claim 11, wherein step (c) comprises combining reference mass spectra of microorganisms that are commonly found in the same location to form combination spectra.

13. The method of claim 11, wherein step (c) comprises combining reference mass spectra that exhibit the closest matches to the sample mass spectrum.

14. The method of any one of claims 11 to 13, wherein selected microorganisms are identified as present in the sample when a match between the sample mass spectrum and a combination spectrum formed from reference mass spectra of the selected microorganisms is better than any match between the sample spectrum and any single reference mass spectrum.

15. A method for identifying microorganisms present in a sample, comprising:
(a) acquiring a mass spectrum of the sample;
(b) obtaining a plurality of reference mass spectra, wherein each reference mass spectrum is a mass spectrum of a known microorganism;
(c) subtracting at least one reference mass spectra from the sample mass spectrum to form a difference spectrum; and
(d) comparing the difference mass spectrum to the reference mass spectra.

16. The method of claim 15, wherein in step (d), a microorganism is identified as present in the sample when a similarity indicator for the match between a reference mass spectrum of that microorganism and the difference mass spectrum is greater than a predetermined minimum value.
